(19) United States
(12) Patent Application Publication
(10) Pub. No.: US 2025/0137923 A1
(43) Pub. Date: May 1, 2025

(54) QUALITY MANAGEMENT APPARATUS, QUALITY MANAGEMENT METHOD, AND STORAGE MEDIUM

(71) Applicant: Konica Minolta, Inc., Tokyo (JP)

(72) Inventors: Junichi JONO, Tokyo (JP); Yuki ONO, Tokyo (JP); Hiroki SHIBATA, Kobe-shi (JP); Makoto OKI, Tokyo (JP)

(21) Appl. No.: 18/918,463

(22) Filed: Oct. 17, 2024

(30) Foreign Application Priority Data
Oct. 31, 2023 (JP) 2023-186525

(51) Int. Cl. G01N 21/3554; G01N 21/3563; G01N 21/359; G01N 33/10

(52) U.S. Cl. CPC G01N 21/3554; G01N 21/3563; G01N 21/359; G01N 33/10

(57) ABSTRACT

Provided is a quality management apparatus for cooked rice coated with a coating agent and conveyed. The quality management apparatus includes: a multispectral camera imager whose imaging wavelength range includes at least a near-infrared region; and a hardware processor. The multispectral camera imager captures a multispectral image of the cooked rice conveyed. The hardware processor acquires and analyzes light absorption information of a plurality of wavelengths related to moisture absorption of the cooked rice from the captured multispectral image of the cooked rice to calculate information indicating a two-dimensional distribution of a moisture content of the cooked rice conveyed.

### CROSS-REFERENCE TO RELATED APPLICATIONS

The entire disclosure of Japanese Patent Application No. 2023-186525 filed on Oct. 31, 2023, is incorporated herein by reference in its entirety.

### TECHNICAL FIELD

The present invention relates to a quality management apparatus, a quality management method, and a storage medium.

### DESCRIPTION OF RELATED ART

In recent years, frozen cooked rice has been widely spread as household food. As the type of frozen cooked rice, for example, a fried rice using cooked rice coated with a coating agent such as oil is known. Cooked rice coated with oil has problems in that, when the amount of moisture contained in the cooked rice is uneven, the cooked rice becomes sticky and the gloss of the cooked rice decreases after thawing.

Japanese Unexamined Patent Publication No. H06-109719 discloses a technology for managing the stickiness and the like of cooked rice. JPH06109719A discloses a quality evaluation method for calculating the water content and the like contained in cooked rice by multiple regression analysis from the wavelength of reflected light reflected from the cooked rice using a near-infrared spectroscopic analyzer.

According to the prior art, the water content, amylose content and the like of cooked rice can be analyzed from the absorption spectrum in the near-infrared region, but the water absorption spectrum cannot be accurately measured for cooked rice coated with oil or the like. Therefore, there is a problem that quality management is difficult for cooked rice coated with oil or the like.

### SUMMARY OF THE INVENTION

Therefore, in order to solve the above-described problem, an object of the present invention is to provide a quality management apparatus that can appropriately control the moisture content of cooked rice coated with a coating agent using a multispectral camera.

A quality management apparatus according to the present invention includes
a quality management apparatus for cooked rice coated with a coating agent and conveyed, the quality management apparatus comprising:
a multispectral camera imager whose imaging wavelength range includes at least a near-infrared region; and a hardware processor,
wherein the multispectral camera imager captures a multispectral image of the cooked rice conveyed,
wherein the hardware processor acquires and analyzes light absorption information of a plurality of wavelengths related to moisture absorption of the cooked rice from the captured multispectral image of the cooked rice to calculate information indicating a two-dimensional distribution of a moisture content of the cooked rice conveyed.

A quality management method for managing quality of cooked rice coated with a coating agent and conveyed according to the present invention includes:
capturing a multispectral image of the conveyed cooked rice using a multispectral camera imager whose wavelength range including at least a near-infrared region; and
acquiring and analyzing light absorption information of a plurality of wavelengths related to moisture absorption of the cooked rice from the captured multispectral image of the cooked rice to calculate information indicating a two-dimensional distribution of a moisture content of the cooked rice conveyed; and
performing feedback control on the moisture content of the cooked rice based on the calculated information.

A non-transitory computer-readable storage medium according to the present invention stores a program for a computer of a quality management apparatus that manages a quality of cooked rice coated with a coating agent and conveyed,
wherein the program causes the computer to:
   acquire and analyze light absorption information of a plurality of wavelengths related to moisture absorption of the cooked rice from a multispectral image of the cooked rice captured by a multispectral camera imager whose imaging wavelength range includes a near-infrared region; and calculate information indicating a two-dimensional distribution of a moisture content of the cooked rice conveyed.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 shows an example of a schematic configuration of a quality management apparatus according to the present embodiment;

FIG. 2 shows an example of an internal configuration of the quality management apparatus, mainly an optical system unit thereof, according to the present embodiment;

FIG. 3 shows a block diagram of the quality management apparatus according to the present embodiment;

FIG. 4 is a flowchart showing an example of a flow of building a classification model and an assessment model used in estimation of a moisture content of cooked rice according to the present embodiment;

FIG. 5 is a flowchart showing an example of an operation of the quality management apparatus in estimation of a moisture content of cooked rice according to the present embodiment; and

FIG. 6 shows an example of a heat map or the like displayed on a display part according to the present embodiment.

### DETAILED DESCRIPTION

### [Configuration Example of Quality Management Apparatus 1]

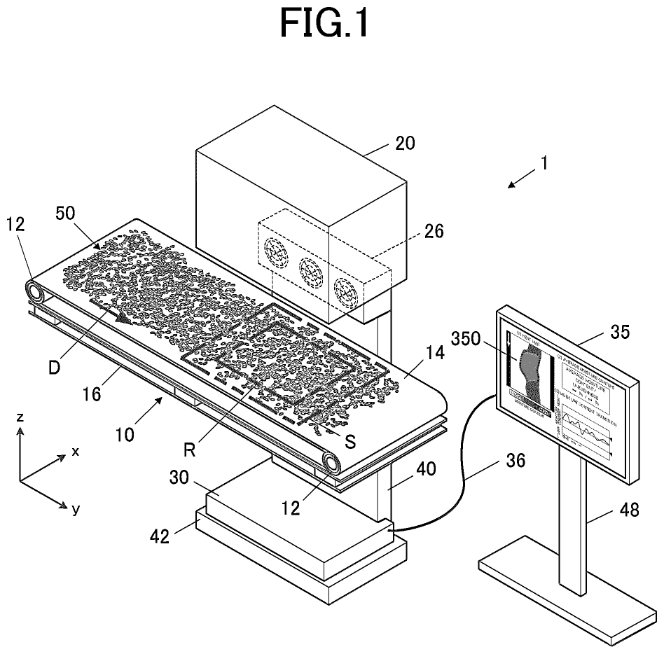

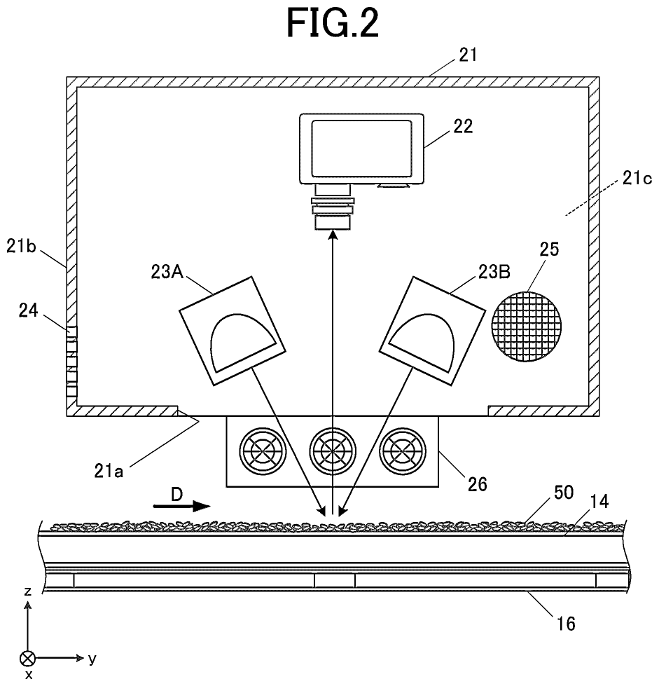

FIG. 1 shows an example of a schematic configuration of a quality management apparatus 1 according to the present embodiment. FIG. 2 shows an example of an internal configuration of the quality management apparatus 1, mainly an optical system unit 20 thereof, according to the present embodiment. In FIG. 1, a width direction of a belt 14 of a conveyance device 10 described later is defined as an x-axis direction, a direction in which the belt 14 extends (a conveyance direction of cooked rice 50) is defined as a y-axis direction, and a direction orthogonal to the x-axis direction and the y-axis direction of the belt 14 is defined as a z-axis direction.

The quality management apparatus 1 is an apparatus for measuring and managing the moisture content of cooked rice of frozen food while the cooked rice is being conveyed before freezing. In the present embodiment, the quality management apparatus 1 is installed in a production line for producing frozen cooked rice. The production line includes, for example, a rice cooking device for cooking rice, a frying device for frying cooked rice, a mixing device for mixing seasonings and the like, and a freezing device for freezing cooked rice. The cooked rice 50 cooked by the rice cooking device is coated with a coating agent such as oil by a frying device or the like. The quality management apparatus 1 can be arranged, for example, on the downstream side of the rice cooking apparatus in order to measure the moisture content of the cooked rice 50 coated with the coating agent.

As shown in FIG. 1, the quality management apparatus 1 includes the optical system unit 20 and a processing device 30. The quality management apparatus 1 is provided with the conveyance device 10 which forms part of the production line and which conveys the cooked rice 50 coated with the coating agent. The conveyance device 10 is, for example, a belt conveyor or the like, and is arranged, for example, on the downstream side of the frying device similarly to the quality management apparatus 1. The conveyance device 10 includes a plurality of rollers 12, an endless belt 14, and a base 16. The plurality of rollers 12 are formed, for example, in pairs and are arranged at predetermined intervals. The rollers 12 are configured to be rotatable by driving of a motor or the like (not shown).

The belt 14 is stretched between the pair of rollers 12, 12, and moves along the conveyance direction D by rotation of the roller 12. On the upper surface of the belt 14, the cooked rice 50 coated with a coating agent is placed to spread over the planar surface. Thus, the cooked rice 50 on the upper surface of the belt 14 is conveyed to an imaging area S by an imager 22. The base 16 is provided below the belt 14 and movably supports the belt 14. The base 16 is configured to be tiltable, so that an inclination angle of the belt 14 can be adjusted. Note that the belt 14 and the base 16 shown in FIG. 1 have a constant length for convenience, and are not limited to the configuration of FIG. 1.

The cooked rice 50 is a food cooked by adding water to rice and boiling or steaming the rice. Each rice grain of the cooked rice 50 is coated with a coating agent in a production process using a frying device, for example. The coating agent includes, for example, at least one of oil, sugar, protein, and egg. Oil and egg are used, for example, in the fried rice of frozen foods and serve as flavour, frying oil, or the like. Oil also functions to mask taste, prevent moisture absorption, avoid contact, suppress consumption of active ingredients, and the like. Sugar is used for preventing deterioration of the cooked rice during chilled distribution and storage. Protein is used for enriching and replenishing nutrients such as amino acids. Note that the coating agent cited in the present embodiment is an example, and other coating agents can also be used.

The optical system unit 20 is attached to an upper end side of a support member 40 extending in the z-axis direction, and is arranged above the belt 14 of the conveyance device 10. The optical system unit 20 is configured to be rotatable and vertically movable with respect to the support member 40 so that the optical system unit 20 can be positioned with respect to the belt 14. As shown in FIG. 2, the optical system unit 20 includes a housing 21, an imager 22, light sources 23A and 23B, an air intake slit 24, an air outlet 25, and an air blower 26.

The housing 21 has a box shape and accommodates the imager 22 and the light sources 23A and 23B therein. An opening 21a is formed on a lower surface of the housing 21 so as to face the belt 14. The opening 21a is an opening for passing the visible light emitted from the light sources 23A and 23B and the reflection light reflected by the cooked rice 50 placed on the upper surface of the belt 14.

The imager 22 is, for example, a multispectral camera. The imager 22 captures an image of the cooked rice 50S conveyed in a conveyance direction D in an imaging area by the belt 14, and acquires a multispectral image of the cooked rice 50. The imager 22, which includes at least a near-infrared region in its imaging wavelength range, disperses light into a plurality of wavelength bands to perform imaging. The imager 22 generates a data cube constituted by superposing layers for each wavelength region obtained by dispersing a two-dimensional plane image of the x-axis and the y-axis of the cooked rice 50 which is an imaging object. The multispectral camera serving as the imager 22 may include a hyperspectral camera. Using the hyperspectral camera, it is possible to capture an image of the cooked rice 50 by dispersing light into finer wavelength bands in comparison to the multispectral camera.

The light sources 23A and 23B include, for example, halogen lamps and LEDs. The light sources 23A and 23B are respectively disposed on the upstream side and the downstream side in the conveyance direction D with respect to the imager 22, and irradiate visible light toward the imaging area S on the upper surface of the belt 14. The visible light emitted from the light sources 23A and 23B is reflected by the cooked rice 50 conveyed by the belt 14, and the reflection light is captured by the imager 22.

The air intake slit 24 is formed, for example, in a front wall 21b of the housing 21. The air intake slit 24 allows outside air to flow into the housing 21. The air outlet 25 is provided, for example, on the downstream side of the air intake slit 24 and is formed in a side wall 21c of the housing 21. The air outlet 25 discharges the air in the housing 21 to the outside. The inside of the housing 21 can be efficiently ventilated by the air intake slits 24 and the air outlet 25, which prevents water vapor flowing into the housing 21 from stagnating and suppresses the occurrence of dew condensation, and the like.

The air blower 26 is disposed, for example, beside the conveyance device 10 and between the upper surface of the belt 14 and the optical system unit 20. In the present embodiment, three fans forming the air blower 26 are arranged along the conveyance direction of the belt 14, but the number of fans is not limited to three. The air blower 26 includes a drive section such as a motor (not shown in the drawings), and is rotated by driving of the drive section, thereby blowing air onto the cooked rice 50 conveyed by the belt 14 from the side. Since the cooked rice 50 can be cooled by this, the inflow of the steam generated from the cooked rice 50 into the housing 21 can be suppressed. The position of the air blower 26 is not limited to that shown in FIG. 2 as long as it can cool the cooked rice 50 on the upper surface of the belt 14.

The processing device 30 is, for example, a personal computer or the like. The processing device 30 is disposed on a placement base 42 provided on the lower end side of the support member 40. The processing device 30 is connected to the conveyance apparatus 10 and the optical system unit 20 via wiring (not shown), and controls the operations of the conveyance device 10 and the optical system unit 20. The processing device 30 acquires spectral data (wavelength information) of a plurality of wavelengths relating to moisture absorption of the cooked rice 50 from the multispectral image of the cooked rice 50 captured by the imager 22. The processing device 30 calculates information indicating a two-dimensional distribution based on the moisture content (amount of water) of the cooked rice 50 by analyzing the spectral data of the acquired multispectral image of the cooked rice 50. The processing device 30 creates a two-dimensional distribution image as information indicating the two-dimensional distribution of the moisture content of the cooked rice 50. Here, the analysis includes, for example, regression analysis. The regression analysis means application of a model built on the basis of the relationship and correlation between explanatory variables such as the spectral data of the multispectral images of the cooked rice 50 and objective variables such as the moisture content of the cooked rice 50. The two-dimensional distribution image is a heat map separately colored with a plurality of tone values or a plurality of colors.

In the present embodiment, the placement base 42 is a fixed type, but the placement base 42 may be a movable type by attaching casters onto the lower side of the placement base 42. With such a movable configuration, when the arrangement of the quality management apparatus 1 is changed in a factory, when the surrounding environment including the quality management apparatus 1 is cleaned, or the like, for example, the quality management apparatus 1 can be freely moved.

The display part 35 is, for example, a liquid crystal display, an organic EL display, or the like. The EL is an abbreviation for Electro Luminescence. The display part 35 is attached onto an upper end side of a stand 48 erected at a position adjacent to the conveyance device 10. The display part 35 is connected to the processing device 30 using a wiring 36 such as an HDMI® cable. The display part 35 may also be wirelessly connected to the processing device 30. The display part 35 shows at least one of the two-dimensional distribution image and the statistical value of the moisture content in a predetermined region of the two-dimensional distribution image on the screen on the basis of the display data output from the processing device 30.

### [Example Block Configuration of Processing Device 30]

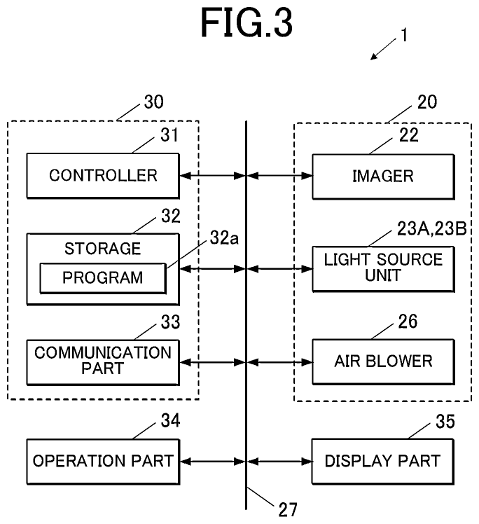

FIG. 3 shows a block diagram of the quality management apparatus 1 according to the present embodiment.

The quality management apparatus 1 includes the processing device 30, the optical system unit 20, an operation part 34, and the display part 35. The processing device 30, the optical system unit 20, the operation part 34, and the display part 35 are connected via, for example, a wiring 27.

The processing device 30 includes a controller 31 (hardware processor), a storage section 32 (storage medium), and a communication part 33.

The controller 31 includes, for example, a processor that performs calculation and control, a memory, and the like. The processor includes, for example, a CPU. CPU is an abbreviation of Central Processing Unit. The controller 31 executes a program 32a stored in a memory such as a RAM, the storage section 32, or the like to realize processing of creating a two-dimensional distribution image of the moisture content of the cooked rice 50 and the like on the basis of the multispectral image of the cooked rice 50.

The controller 31 may include an electronic circuit such as an ASIC or an FPGA. ASIC is an abbreviation of Application Specific Integrated Circuit. FPGA is an abbreviation for Field Programmable Gate Array.

In the present embodiment, the processor of the controller 31 implements the following functions by executing the program 32a stored in the storage section 32 and the like. The controller 31 acquires spectral data of a plurality of wavelengths related to the moisture absorption of the cooked rice 50 spread planarly and conveyed from the multi-spectral camera image of the cooked rice 50 which is captured by the imager 22 whose imaging wavelength range includes at least a near-infrared region. The controller 31 performs regression analysis using the acquired spectral data, and creates a heat map indicating the moisture content distribution of the cooked rice 50 which is spread planarly and conveyed. The controller 31 may calculate a statistical value of the moisture content in a predetermined region of the created heat map. The controller 31 causes the display part 35 to show at least one of the heat map and the statistical value of the moisture content in a predetermined area of the heat map.

The storage section 32 includes any storage module such as an HDD, an SSD, a ROM, and a RAM, for example. HDD is an abbreviation of Hard Disk Drive. SSD is an abbreviation for Solid State Drive. ROM is an abbreviation of Read Only Memory. The storage section 32 stores, for example, a system program, an application program, and various types of data. To be more specific, the storage section 32 stores the program 32a for executing the processing of creating a two-dimensional distribution image of the moisture content of the cooked rice 50 based on the multispectral image of the cooked rice 50. The storage section 32 may store a machine learning model (learned model) for deducing the moisture content of the cooked rice 50 from the multispectral image of the cooked rice 50.

The communicator 33 includes, for example, a communication module including an NIC, a receiver, and a transmitter. NIC is an abbreviation for Network Interface Card. The communication part 33 communicates various kinds of information and data with an external device or the like connected via a network such as the Internet.

The operation part 34 includes, for example, a mouse, a keyboard, a switch, and a button. The operation part 34 may be, for example, a touch screen integrally combined with the display part 35, or may be an interface that receives voice input. The operation part 34 receives a command according to various input operations from a user, converts the received command into an operation signal, and outputs the operation signal to the controller 31. Specifically, the operation part 34 receives a command to turn on or off a mode for measuring the moisture content of the cooked rice 50 or the like, a specification of a region for displaying a heat map indicating the moisture content distribution of the cooked rice 50, and the like.

Based on the display data output from the controller 31, the display part 35 shows a two-dimensional distribution image of the cooked rice 50, a GUI for receiving various input operations from the user, and the like. GUI is an abbreviation for Graphical User Interface.

The optical system unit 20 includes the imager 22, the light sources 23A and 23B, and the air blower 26. The imager 22 captures images of the cooked rice 50 on the upper surface of the belt 14 based on the control of the controller 31. The light sources 23A and 23B emits light to the cooked rice 50 on the upper surface of the belt 14 with light based on the control of the controller 31. The air blower 26 is rotated by driving of the drive section such as a motor under the control of the controller 31. The optical system unit 20 may be provided with a controller such as a CPU that performs calculation and control, and the controller 31 of the optical system unit 20 and the controller 31 of the processing device 30 may perform processing in cooperation with each other.

### [Example of Building Machine Learning Model]

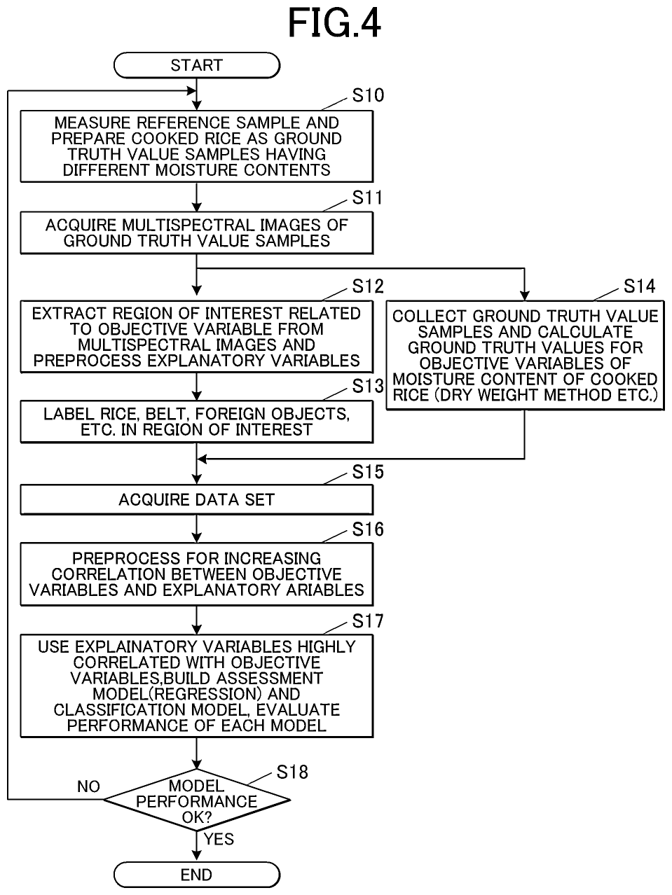

Next, a flow of building a machine learning model used for estimating the moisture content of the cooked rice 50 from the multispectral image of the cooked rice 50 will be described. FIG. 4 is a flowchart showing an example of a flow of a case where a classification model and an assessment model are built as the machine learning models. In the present embodiment, an example in which a machine learning model is built using the quality management apparatus 1 and a learning device (not shown) will be described, but the present invention is not limited thereto. For example, another imaging device or conveyance device may be used. The processing device 30 of the quality management apparatus 1 may be used as the learning device. The learning device is, for example, a computer, and includes a processor or the like including a CPU, a GPU, and the like. GPU is an abbreviation for Graphics Processing Unit.

First, as ground truth value samples, a plurality of varieties of cooked rice having different moisture contents are produced as preparation necessary for building a model (Step S10). As a ground truth value sample, cooked rice coated with a coating agent such as oil is used. The moisture contents of the prepared cooked rice having different moisture contents are measured in order by the quality management apparatus 1. In addition, a sample as a reference (hereinafter, reference sample) is measured. In capturing multispectral images, illumination light emitted from the light sources 23A and 23B is absorbed and scattered by an object to be captured such as the cooked rice 50, and images are generated based on the signal intensity acquired through the optical path to the imager 22. Therefore, a signal from an object whose absorption and scattering properties are known is acquired in advance, and the signal is used as a reference value, thus allowing more accurate water content estimation. In estimating the moisture content, a material that does not contain moisture or hardly contains moisture is used preferably as the reference sample, and for example, a standard diffuse reflector having stable optical characteristics is used desirably. Further, the measurement object itself may be used as the reference sample. That is, as the reference sample, a sample obtained by completely drying the cooked rice 50 in advance and finely crushing or leveling the cooked rice 50 in order to suppress variations in characteristics due to the surface shape may be used. When the signal intensity at the time of measurement of the reference sample is set as a reference value and the ratio of the signal intensity at the time of actual measurement of the moisture content of the cooked rice 50 to the reference value is calculated, the ratio is converted into the reflectance. Stable acquisition of the signal intensity at the time of measurement of the reference sample facilitates comparison between a plurality of varieties of cooked rice having different moisture contents or between data on different measurement dates or under different conditions.

Next, the imager 22 of the quality management apparatus 1 captures an image of each variety of cooked rice that is a ground truth value sample conveyed by the belt 14, and acquires a multispectral image of each variety of cooked rice (step S11). When the moisture contents are different between the plurality of varieties of cooked rice, the spectral intensity (absorptivity) of a specific wavelength relating to the moisture of the cooked rice varies. Also, in the multispectral images, the spectral intensity of the wavelength associated with the coating agent, such as oil, also changes.

Next, the learning device extracts a region of interest (ROI) R related to an objective variable, the moisture content of the cooked rice, from the acquired multispectral image of the cooked rice (Step S12). As shown in FIG. 1, the region of interest R indicates, for example, a partial region of the multispectral image.

In addition, the learning device preprocesses the multispectral image of the cooked rice, which is an explanatory variable. The preprocessing includes, for example, noise processing and correction processing.

Next, labeling is performed on the region of interest R extracted from the multispectral image (Step S13). Specifically, the region of interest R is classified into each region of cooked rice, a belt, a foreign substance, and the like, and each classified region is labeled by attaching a ground truth label. For example, when the moisture content of the cooked rice 50 in a production process is estimated, it is relatively clear where an information amount to be estimated is present in the multispectral image. Therefore, the initial labeling may be basically done by the user. The labeling result may lack accuracy due to oversight or misrecognition by the user. In such a case, the labeling result and principal component analysis (PCA) or the like may be further used in combination to specify an image region having an information amount similar to that of the labeled region, thereby extending or correcting the labeling. Alternatively, the multi-spectral image to which the labeling information is added may be used as train data, and the labeling may be strengthened through machine learning or the like. Furthermore, the user might not be able to determine where in the multispectral image a target to be labeled is included. The user cannot determine where the target is, for example, in a case where the cooked rice is uniformly present in the entire visual field or a case where there is a foreign substance that cannot be found by the user. In such a case, the labeling may be mechanically performed using a clustering method such as a k-means method. Alternatively, using a plurality of multispectral images that appear normal to the user's eyes, a multispectral image including an abnormal value may be detected through machine learning for labeling.

In parallel with the classification processing of the region of interest R, a ground truth value is calculated for the moisture content (amount of water) of the cooked rice, which is an objective variable for each of the created ground truth value samples (Step S14). Specifically, by performing a dry weight method on each of the varieties of cooked rice having different moisture contents, the moisture content of each variety of cooked rice is calculated from the weight of the cooked rice before drying and the weight of the cooked rice after drying. Thus, the moisture content of each variety of cooked rice can be calculated from the amount of moisture in each variety of cooked rice.

Next, the learning device acquires a dataset for building a machine learning model (Step S15). The data set includes explanatory variables and objective variables. Specifically, the data set used to build an assessment model includes the spectral data of the multispectral image of the cooked rice and the moisture content of the cooked rice in predetermined ground truth value samples. The data set used to build a classification model includes the multispectral image of the cooked rice and the ground truth labels of the cooked rice, belt, foreign matter, and the like classified in the region of interest R in predetermined ground truth value samples. In one multi-spectral image, there may be at least one or more regions of interest R associated with a ground truth value of a water content and at least one or more regions of interest R indicating a ground truth label of a foreign substance or the like. In this case, the multispectral image may be used as both an explanatory variable for the assessment model and an explanatory variable for the classification model.

Furthermore, the region of interest R indicating the ground truth value of the moisture content used for the data set of the assessment model and the region of interest R indicating the cooked rice used for the data set of the classification model may be the same. Further, although the main explanatory variables of each model are the spectral data in the multi-spectral image, external elements which may have a relationship with the objective variables, for example, a measurable environmental factor such as humidity, a material formulation, a manageable parameter of a manufacturing condition, or the like may be used supplementally for the purpose of improving regression or classification accuracy. As described in Step S10, the spectral data serving as the explanatory variables may be reflectance indicating a ratio to the reference value that is signal intensity at the time of measurement of the reference sample. Further, in order to increase the correlation with the objective variable, for example, values obtained by logarithmic transformation or differentiation may be used.

Next, the learning device preprocesses the acquired dataset in order to increase the correlativity between the objective variables and the explanatory variables (Step S16). For example, the learning device applies baseline correction or the like as preprocessing to the acquired data set. The pre-processing may include processing such as standardization and regularization.

Next, the learning device builds an assessment model and a classification model using explanatory variables highly correlated with the objective variable, and evaluates the performance of each of the built models (Step S17). Specifically, the learning device trains each model using the acquired data set. Thus, for example, an assessment model for estimating the moisture content of predetermined cooked rice can be generated from a multispectral image of the cooked rice. In addition, it is possible to generate a classification model for distinguishing a region of predetermined cooked rice from other backgrounds and the like from a multispectral image of the predetermined cooked rice.

The assessment model can be built by a method such as linear regression, multiple regression analysis, a support vector machine, or a decision tree. As more specific methods of the linear regression and the multiple regression analysis, a least squares method, principal component regression, partial least squares regression, ridge regression, ElasticNet, and the like are effective. The classification model can be built by a method such as a support vector machine or a decision tree. As the classification model, a k-means method, a k-nearest neighbor method, logistic regression, linear discriminant classification, partial least squares discriminant analysis, a maximum likelihood classification method, random forest, and the like are also effective. Furthermore, the assessment model and the classification model may be generated by a method such as a neural network.

The learning device determines whether the built models have any problem in their performances (Step S18). Specifically, the learning device evaluates the performances of the built models using verification data. If the learning device determines that the built model has satisfactory performances (Step S18: YES), the learning device ends the model building processing. On the other hand, when determining that a built model has a problem in its performances (Step S18: NO), the learning device returns to Step S10 and builds a model again.

### [Operation Example of Quality Management Apparatus 1]

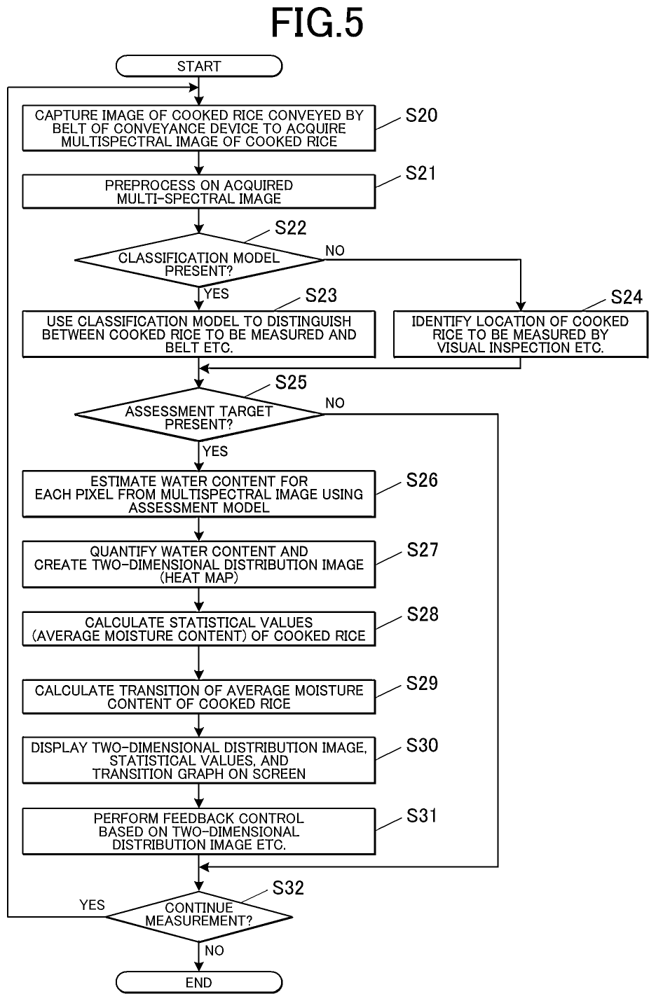

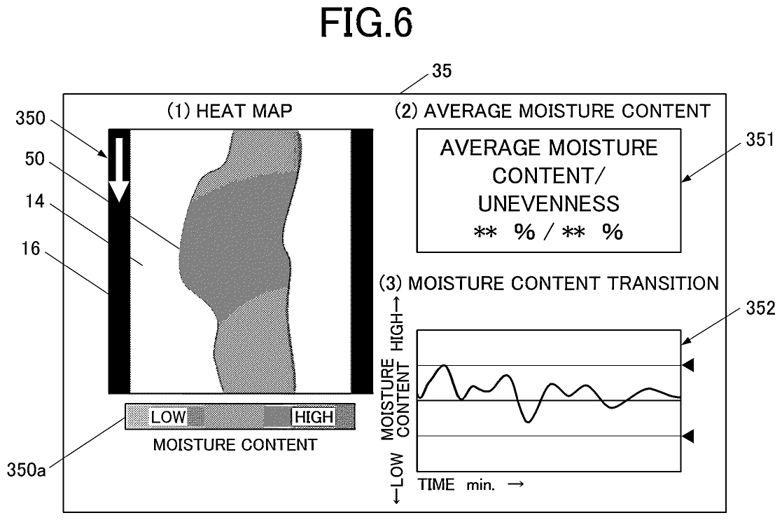

Next, a flow of the quality management apparatus 1 in measuring and controlling the moisture content of the cooked rice 50 used for an actual frozen food will be described. FIG. 5 is a flowchart showing an example of processing of a quality management method performed by the quality management apparatus 1 according to the present embodiment. FIG. 6 shows an example of the heat map 350 and the like displayed on the display part 35 according to the present embodiment.

The imager 22 captures an image of the cooked rice 50 placed on the upper surface of the belt 14 and conveyed by the conveyance device 10 and acquires a multispectral image of the cooked rice 50 (Step S20). Step S20 corresponds to an imaging step. The multispectral image includes, in addition to the cooked rice 50, the belt 14, a foreign substance mixed in the cooked rice 50, and the like.

The controller 31 of the processing device 30 acquires the multispectral image of the cooked rice 50 captured by the imager 22 and preprocesses the acquired multispectral image (Step S21). Specifically, the controller 31 applies, on the acquired multispectral image, the preprocessing applied when the assessment model or the like is built. The preprocessing includes noise processing and the like. The controller 31 acquires spectral data of a plurality of wavelengths relating to moisture absorption of the cooked rice 50 from the multispectral image of the cooked rice 50. In the spectral data, the spectral intensity (absorptivity) of a specific wavelength relating to the moisture of the cooked rice 50 changes according to the moisture content of the cooked rice 50.

The controller 31 determines whether or not there is a classification model (Step S22). If the controller 31 determines that there is a classification model (Step S22: YES), the controller 31 proceeds to Step S23. In this case, the controller 31 deduces the regions of the cooked rice 50, the belt 14, the foreign substance, and the like in the multispectral image using the classification model (step S23). Specifically, the controller 31 inputs the acquired multispectral image into the classification model to acquire output data in which the cooked rice 50 as the measurement target and the other components such as the belt 14 are distinguished from each other in the multispectral image. The controller 31 may limit the range of the multispectral images to be input to the classification model as necessary.

On the other hand, if the controller 31 determines that there is no classification model (Step S22: NO), the controller 31 proceeds to Step S24. In this case, the controller 31 shows the multispectral image on the screen of the display part 35. The user views the multispectral image displayed on the screen and specifies the region of the cooked rice 50 using the operation part 34 or the like Step S24). A specifying method includes, for example, designation of the region by a mouse or the like, designation of the region by a touch operation on a touch screen, and the like.

The controller 31 determines whether or not there is an assessment target (Step S25). Specifically, the controller 31 determines whether or not the cooked rice 50 to be assessed exists in the acquired multispectral image. When determining that there is an assessment target in the multispectral image (Step S25: YES), the controller 31 proceeds to Step S26. For example, it is when determining in Step S23 or the like that there is the cooked rice 50 in the multispectral image. On the other hand, when determining that there is no assessment target in the multispectral image (Step S25: NO), the controller 31 proceeds to Step S32.

The controller 31 estimates the moisture content of the conveyed cooked rice 50 from the multispectral image of the cooked rice 50, using the assessment model (Step S26). Specifically, the controller 31 acquires, as output data, the moisture content of each pixel of the multispectral image of the cooked rice 50 by inputting the acquired multispectral image of the cooked rice 50 into the assessment model.

The controller 31 quantifies the water content of each pixel of the estimated multispectral image to calculate information indicating the two-dimensional distribution of the water content of the conveyed cooked rice 50, and creates a two-dimensional distribution image (Step S27). Step S27 corresponds to a calculation step. Specifically, the controller 31 performs calculation for converting the moisture content of each pixel of the multispectral image into a luminance value, thereby creating a heat map in which pixels are separately colored with a plurality of tone values or a plurality of colors. The luminance values may be calculated using, for example, a conversion formula, a conversion coefficient, or the like for converting preset moisture contents into respective luminance values. The controller 31 outputs display data corresponding to the created two-dimensional distribution image to the display part 35.

The controller 31 calculates a statistical value such as an average moisture content of the cooked rice 50 using the estimated moisture content of each pixel of the multispectral image of the cooked rice 50 (step S28). Specifically, the controller 31 calculates the average moisture content based on the moisture contents of all the pixels in the predetermined region of the multispectral image. The controller 31 outputs display data corresponding to the calculated average moisture content to the display part 35. The statistical value may be other than the average moisture content, and may be, for example, a median value or a standard deviation of the moisture content.

The controller 31 creates a transition graph of the average moisture content by adding the time-series data to the calculated average moisture content of the cooked rice 50 (Step S29). The transition graph has a temporal resolution of, for example, several minutes with intervals plotted. The controller 31 outputs display data corresponding to the created transition graph to the display part 35.

The display part 35 shows the heat map, the average moisture content of the cooked rice 50, and the transition graph on the screen based on the various display data output from the controller 31 (Step S30). Step S30 corresponds to a display step. Specifically, as shown in FIG. 6, the display part 35 shows the heat map 350 on the left side of the screen, the statistical value 351 such as the average moisture content of the cooked rice 50 on the upper right side of the screen, and the transition graph 352 on the lower right side of the screen.

The heat map 350 is an image in which the cooked rice 50 on the upper surface of the belt 14 is shown two-dimensionally in the x-axis direction and the y-axis direction, colored with a plurality of gradation values or a plurality of colors in accordance with the moisture content of the cooked rice 50 in the imaging area S. The heat map 350 includes the belt 14, the cooked rice 50 placed on the belt 14, and the base 16 that supports the belt 14. Shown below the heat map 350 is a tone display part 350a which indicates a correspondence between the moisture contents of the cooked rice 50 and the tone values (light and shade). In the present embodiment, a region of the cooked rice 50 having a higher moisture content is indicated by a darker blue color, and a region of the cooked rice 50 having a lower moisture content is indicated by a lighter blue color. In FIG. 6, the shade of blue is illustrated by the shade of gray for convenience. In the heat map 350, an upstream side in the movement direction of the belt 14 (y-axis direction) is shown on an upper side, and a downstream side in the moving direction of the belt 14 is shown on a lower side. In the heat map 350, the width direction of the belt 14 is defined as the left-right direction (x-axis direction). According to the heat map 350, it can be seen that, in the cooked rice 50 planarly spread on the upper surface of the belt 14, the moisture content of the cooked rice 50 in the central region is higher than the moisture content of the cooked rice 50 in the upper region and the lower region.

In the heat map 350, a predetermined area to be displayed on the display part 35 may be specified in advance by a user through the operation part 34 or the like. In designating the predetermined area, for example, an operation of a mouse, a touch operation on a touch screen, and the like may be used as a method. When a predetermined area of the heat map 350 being displayed is specified by the user, the controller 31 may display the specified predetermined area in an enlarged manner or in another window. The controller 31 may display a heat map of cooked rice acquired in the past side by side in a region adjacent to the heat map 350 of the current cooked rice 50. In this case, a heat map indicating an ideal moisture distribution of the cooked rice may be displayed instead of the past heat map of the cooked rice.

The calculated average moisture content and standard deviation (variation) of the cooked rice 50 are displayed as the statistical value 351. In the present embodiment, the average moisture content and the standard deviation are displayed as the statistical values of the moisture content of the cooked rice 50, but the present invention is not limited thereto. A median value may be displayed as the statistical value of the moisture content of the cooked rice 50.

In the transition graph 352, the vertical axis represents the water content and the horizontal axis represents time. For example, an upper limit value and a lower limit value may be displayed as threshold values in the transition graph 352. This allows the user to visually grasp whether the moisture content of the cooked rice 50 currently being conveyed is optimal or not.

In the present embodiment, the heat map 350, the statistical value 351 such as the average moisture content of the cooked rice 50, and the transition graph 352 are displayed on the display part 35, but the layout is not limited to that shown in FIG. 6. For example, only the heat map 350 may be displayed on the display part 35, or the heat map 350 and one of the statistical value 351 and the transition graph 352 may be displayed on the display part 35.

A worker on site can check, in real time, information on the moisture content and the like of the cooked rice 50 conveyed on the belt 14 by looking at the heat map 350 and the like on the display part 35. For example, when the moisture content of the cooked rice 50 is biased or varies in the heat map 350 of the display part 35, the worker adjusts various conditions in the production process of the cooked rice 50. Examples of the various conditions include a rice cooking time, a rice cooking temperature, and a water amount in the rice cooking device, and a time of stirring processing of the rice after the rice cooking. The stirring processing may be performed by a device other than the rice cooking device. Other conditions include, for example, a cooking time in the frying device, a cooking temperature, a coating amount of the coating agent on the cooked rice 50, and the like. The processing device 30 acquires correction information corresponding to each condition for adjusting the moisture content (amount of water) of the cooked rice 50 input by the worker or the like. The processing device 30 executes feedback control for adjusting various conditions in the production process on the basis of the acquired correction information so that the moisture content of the cooked rice 50 reaches a target value (Step S31). Step S31 corresponds to a control step. The feedback control may be performed by an arithmetic device different from the processing device 30.

An example in which the quality management apparatus 1 shown in FIG. 1 and the like is provided with the display part 35 that shows the heat map 350 and the like has been described, but the present invention is not limited thereto. For example, the quality management apparatus 1 may be configured without the display part 35. That is, the processing device 30 or the like may automatically adjust the moisture content of the cooked rice 50 without involving the operation by the worker or the like. In such a case, the processing device 30 calculates correction information with which the moisture content of the cooked rice 50 reaches a target value using information such as the heat map 350 of the calculated moisture content of the cooked rice 50, and performs feedback control using the calculated correction information.

Further, in the quality management apparatus 1, the configuration with the display part 35 for displaying the heat map 350 and the like of the moisture content of the cooked rice 50 may be used in combination with the automatic adjustment by the processing device 30. In such a case, a worker or the like may be able to monitor the heat map 350 displayed on the display part 35 and manually adjust various conditions in the production process in addition to automatic adjustment by the processing device 30.

The controller 31 determines whether to continue the measurement of the moisture content of the cooked rice 50 to be conveyed (Step S32). For example, when determining that an operation for ending the measurement has been performed by a worker or the like (Step S32: YES), the controller 31 ends the series of measurements. On the other hand, when determining that the operation for ending the measurement has not been performed by the worker or the like (Step S32: NO), the controller 31 returns to Step S20 and continues the measurement of the moisture content of the cooked rice 50 to be conveyed.

According to the present embodiment, the heat map 350 indicating the two-dimensional distribution of the moisture content of the cooked rice 50 coated with the coating agent in the imaging area S is created, and the created heat map 350 is displayed on the display part 35. This makes it possible to accurately grasp the moisture content distribution of adjacent regions and close regions of the cooked rice 50 before freezing which is being conveyed by the belt 14. Thus, various conditions in the production process can be adjusted according to the moisture content of each region of the cooked rice 50 planarly spread and conveyed on the belt 14. For example, it is possible to adjust the amount of moisture in cooking rice, a time for drying rice, and the amount of coating of the coating agent. As a result, since the moisture content of the cooked rice 50 can be optimally managed in the cooked rice 50 before freezing, the stickiness of the cooked rice 50 after thawing can be avoided, and the gloss of the cooked rice 50 can also be improved.

Furthermore, according to the present embodiment, the statistical value 351 such as the average moisture content of the cooked rice 50 coated with the coating agent and the transition graph 352 of the moisture content of the cooked rice 50 are displayed on the display part 35, which makes it possible to grasp more accurately the moisture amount distribution in adjacent regions or close regions of the cooked rice 50 before freezing.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

1. A quality management apparatus for cooked rice coated with a coating agent and conveyed, the quality management apparatus comprising:
a multispectral camera imager whose imaging wavelength range includes at least a near-infrared region; and a hardware processor,
wherein the multispectral camera imager captures a multispectral image of the cooked rice conveyed,
wherein the hardware processor acquires and analyzes light absorption information of a plurality of wavelengths related to moisture absorption of the cooked rice from the captured multispectral image of the cooked rice to calculate information indicating a two-dimensional distribution of a moisture content of the cooked rice conveyed.

2. The quality management apparatus according to claim 1,
wherein the hardware processor creates a two-dimensional distribution image as the information indicating the two-dimensional distribution of the moisture content of the cooked rice, and causes a display part to show the two-dimensional distribution image and/or a statistical value of the moisture content in a predetermined region of the two-dimensional distribution image.

3. The quality management apparatus according to claim 1,
wherein the hardware processor acquires the light absorption information on the moisture absorption of the cooked rice of the plurality of wavelengths and performs regression analysis.

4. The quality management apparatus according to claim 1, further comprising
a display part which shows a two-dimensional distribution image and/or a statistical value of the moisture content in a predetermined region of the two-dimensional distribution image as the information indicating the calculated two-dimensional distribution.

5. The quality management apparatus according to claim 1,
wherein the coating agent is at least one or more of oil, sugar, protein, and egg.

6. The quality management apparatus according to claim 2
wherein the two-dimensional distribution image is a heat map colored with a plurality of tone values or a plurality of colors.

7. The quality management apparatus according to claim 2,
wherein the hardware processor further causes the display part to show a transition of the statistical value.

8. The quality management apparatus according to claim 2,
wherein the statistical value is at least one of an average value, a median value, and a standard deviation.

9. The quality management apparatus according to claim 2, further comprising:
an operation part with which a user can specify the predetermined region of the two-dimensional distribution image.

10. The quality management apparatus according to claim 2,
wherein the hardware processor further causes the display part to show a two-dimensional distribution image of a moisture content of cooked rice in past or a two-dimensional distribution image of a moisture content of ideal cooked rice.

11. The quality management apparatus according to claim 1,
wherein the multispectral camera imager includes a hyperspectral camera.

12. A quality management method for managing quality of cooked rice coated with a coating agent and conveyed, the method comprising:
capturing a multispectral image of the conveyed cooked rice using a multispectral camera imager whose wavelength range including at least a near-infrared region; and
acquiring and analyzing light absorption information of a plurality of wavelengths related to moisture absorption of the cooked rice from the captured multispectral image of the cooked rice to calculate information indicating a two-dimensional distribution of a moisture content of the cooked rice conveyed; and
performing feedback control on the moisture content of the cooked rice based on the calculated information.

13. A non-transitory computer-readable storage medium storing a program for a computer of a quality management apparatus that manages a quality of cooked rice coated with a coating agent and conveyed,
wherein the program causes the computer to:
acquire and analyze light absorption information of a plurality of wavelengths related to moisture absorption of the cooked rice from a multispectral image of the cooked rice captured by a multispectral camera imager whose imaging wavelength range includes a near-infrared region to calculate information indicating a two-dimensional distribution of a moisture content of the cooked rice conveyed.
